(19) United States
(12) Patent Application Publication
(10) Pub. No.: US 2022/0054084 A1
(43) Pub. Date: Feb. 24, 2022

(54) SYSTEMS AND METHODS FOR PHYSIOLOGY MONITORING KNEE BRACE

(71) Applicant: MYANT INC., Toronto (CA)

(72) Inventors: Tony CHAHINE, Toronto (CA); Milad ALIZADEH-MEGHRAZI, Toronto (CA); Saransh Bhushan JAIN, Toronto (CA)

(21) Appl. No.: 17/421,173

(22) PCT Filed: Jan. 7, 2020
(86) PCT No.: PCT/CA2020/050012

§ 371 Date: Jul. 7, 2021

Related U.S. Application Data
(60) Provisional application No. 62/789,359, filed on Jan. 7, 2019.

(51) Int. Cl. A61B 5/00; A61F 5/01; A61F 7/00; A61N 1/36; A61B 5/389; A61B 5/11

(52) U.S. Cl. CPC A61B 5/6812; A61F 2007/0228; A61F 5/0109; A61F 7/007; A61N 1/36021; A61B 5/002; A61B 5/389; A61B 5/11; A61B 5/4836; A61N 1/36031; A61B 2562/0261; A61B 2562/0219; A61B 2560/0214; A61F 2007/0071; A61F 2007/0042; A61B 5/4585

(57) ABSTRACT

A knee brace is provided for monitoring the knee. The knee brace includes a dock for removably receiving a control module. The control module is electrically coupled with the dock when the control module is received in the dock. The knee brace includes a sensor for measuring a physiological state of the wearer. The sensor is electrically coupled to the control module by way of the first conductive path when the control module is received in the dock.

### CROSS REFERENCE TO RELATED APPLICATIONS

This application also claims priority from U.S. provisional patent application No. 62/789359, filed on Jan. 7, 2019; the entire contents of which are hereby incorporated by reference herein

### FIELD

The present disclosure generally relates to the field of knee braces and in particular knee braces with physiological monitoring and pain relief capabilities.

### BACKGROUND

The knee is the largest joint and one of the most important joints in the human body. It plays an essential role in movement related to carrying the body weight in horizontal (running and walking) and vertical (jumping) directions. As such, high levels of physical activity may lead to the development of knee problems over time, including for example osteoarthritis.

Physical fitness is also integrally related to the development of knee problems. Activity such as climbing stairs may cause pain for someone who is physically unfit due to patellofemoral compression, whereas others may experience no pain. Even for fit individuals, a person may experiences pain at a different time. Obesity is another major contributor to knee pain.

Various knee injuries, such as, torn ligaments or cartilage, bone fractures, or displacement of the kneecap from normal positions, may require knee treatment including surgical interventions. During non-operative and postoperative rehabilitation, patient can experience high levels of pain requiring pain management in addition to knee support.

Accordingly, improved knee braces are needed for proper monitoring of the knee. Improved knee braces are also needed for pain relief.

### SUMMARY

Monitoring the physiological functions of the knee allows for early detection of knee problems, as well as prevention or mitigation of complications.

In one aspect of the disclosure, there is provided a knee brace for monitoring a knee of a wearer. The knee brace includes a textile article shaped to cover at least part of a knee of the wearer. The textile article includes conductive yarn arranged to define a first conductive path. The knee brace includes a dock for removably receiving a control module. The control module is electrically coupled with the dock when the control module is received in the dock. The knee brace includes a sensor for measuring a physiological state of the wearer. The sensor is electrically coupled to the control module by way of the first conductive path when the control module is received in the dock.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the instant disclosure.

### DESCRIPTION OF THE FIGURES

Reference is now made to the accompanying drawings, in which:

FIG. 1 is a perspective view of a knee brace and flexible battery belt that is used for monitoring a knee;

FIG. 2 is a front view of a module and battery assembly of a battery belt;

FIG. 3 is a perspective view of a module and battery assembly of a battery belt;

FIG. 4 shows a perspective view, front view, and rear view of a knee brace having an elastic strap;

FIG. 5 is a schematic depiction of a module including a computing device, one or more communication ports, and a transceiver;

FIG. 6 shows a knee brace including a heating element, a stimulator and a dock

FIG. 7 shows a knee brace including a stretch sensor, a heating element, a stimulator and a dock;

FIG. 8 shows a knee brace including inertial measurement units (IMUs) and electromyography (EMG) sensors;

FIG. 9 shows the orientation and axis of the IMUs of FIG. 7 worn by a wearer when the wearer's knee is at two different positions; and

FIG. 10 shows a knee brace including IMUs and EMG sensors connected to a module.

### DETAILED DESCRIPTION

The following description discloses knee braces, systems and methods useful for monitoring a knee. A knee brace is disclosed herein that may be configured to monitor a knee of a wearer of the knee brace by measuring a physiological state of the knee using a sensor. The knee brace be configured to provide treatment to the knee in the form of heat treatment or stimulation treatment. The knee brace may include a dock configured to receive a module that activates the monitoring or treatment function of the knee brace.

The term “connected” or “coupled to” as used herein may include both direct coupling (in which two elements that are coupled to each other contact each other) and indirect coupling (in which at least one additional element is located between the two elements).

As used herein, “textile” refers to any material made or formed by manipulating natural or artificial fibres to interlace to create an organized network of fibres. Generally, textiles are formed using yarn, where yarn refers to a long continuous length of a plurality of fibres that have been interlocked (i.e. fitting into each other, as if twined together, or twisted together).

As used herein, an “electrical component” refers to a sensor, or a component for delivering a treatment to a knee. Examples of an electrical component include, but are not limited to, motion sensor such as a stretch sensor or an inertial measurement unit, current sensor, temperature sensor, pulse detector, heating element, electrical stimulator, temperature regulator, or pressure applicator.

As used herein, “interlace” refers to fibres or yarn (either artificial or natural) crossing over and/or under one another in an organized fashion, typically alternately over and under one another, in a layer. When interlaced, adjacent fibres touch each other at intersection points (e.g. points where one fibre crosses over or under another fibre). In one example, first fibres extending in a first direction can be interlaced with second fibres extending laterally or transverse to the fibres extending in the first connection. In another example, the second fibres can extend laterally at 90° from the first fibres when interlaced with the first fibres. Interlaced fibres extending in a sheet can be referred to as a network of fibres.

As used herein “integrated” or “integrally” refers to combining, coordinating or otherwise bringing together separate elements so as to provide a harmonious, consistent, interrelated whole. In the context of a textile, a textile can have various sections comprising networks of fibres with different structural properties. For example, a textile can have a section comprising a network of conductive fibres and a section comprising a network of non-conductive fibres. Two or more sections comprising networks of fibres are said to be “integrated” together into a textile (or “integrally formed”) when at least one fibre of one network is interlaced with at least one fibre of the other network such that the two networks form a layer of the textile. Further, when integrated, two sections of a textile can also be described as being substantially inseparable from the textile. Here, “substantially inseparable” refers to the notion that separation of the sections of the textile from each other results in disassembly or destruction of the textile itself.

Aspects of various embodiments are described through reference to the drawings.

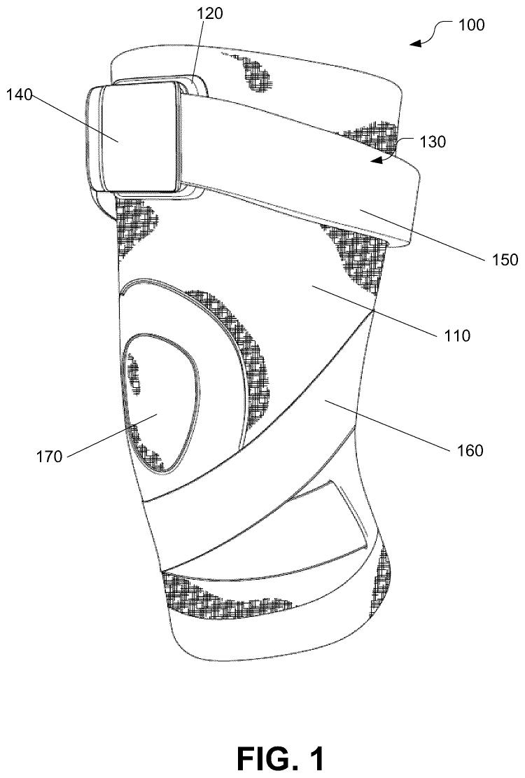

FIG. 1 depicts a perspective view of knee brace 100 and flexible battery belt 130 that is used for monitoring a knee. Knee brace 100 includes textile article 110 that is shaped to cover at least part of a knee of a wearer of knee brace 100. In some embodiments, textile article 110 may define aperture 170 that exposes a kneecap of the user when textile article 110 is worn by the user. It should be appreciated that textile article 110 may vary in size based on a size of the knee of the wearer.

In some embodiments, textile article 110 may be formed of a knitted textile. In some embodiments, textile article 110 may be formed of other textile forms and/or techniques such as weaving, knitting (warp, weft, etc.) or the like. In some embodiments, textile article 110 includes any one of a knitted textile, a woven textile, a cut and sewn textile, a knitted fabric, a non-knitted fabric, in any combination and/or permutation thereof. Example structures and interlacing techniques of textiles formed by knitting and weaving are disclosed in U.S. Ser. No. 15/267,818, the entire contents of which are herein incorporated by reference.

Different sections of a textile can be integrally formed into a layer to utilize different structural properties of different types of fibres. For example, conductive fibres can be manipulated to form networks of conductive fibres and non-conductive fibres can be manipulated to form networks of non-conductive fibers. These networks of fibres can comprise different sections of a textile by integrating the networks of fibres into a layer of the textile. The networks of conductive fibres can form one or more conductive pathways that electrically connect sensors and actuators (such as for example, stimulators 633 or heating elements 632 detailed below) embedded into textile article 110, for conveying data and/or power to and/or from these electrical components.

In some embodiments, multiple layers of textile can also be stacked upon each other to provide a multi-layer textile.

In some examples, conductive fabric (e.g. group of conductive fibres) can be knit along with (e.g. to be integral with) the base fabric (e.g. surface) in a layer. Such knitting may be performed using a circular knit machine or a flat bed knit machine, or the like, from a vendor such as Santoni or Stoll.

Knee brace 100 may include dock 120 that may be attached to textile article 110. As depicted in FIG. 1, dock 120 may be configured to receive module 140 (discussed in further detail below). Dock 120 may be positioned above the knee of the user. It is understood that dock 120 may also be located at other areas of knee brace 100 that do not interfere with movement of the knee.

Knee brace 100 may include one or more electrical components connected to dock 120. The one or more electrical components may be inlaid within textile article 110 and therefore are not visible in FIG. 1. Textile article 110 may include conductive yarn to define conductive paths between the electrical components and dock 120. Dock 120 may include terminals made of conductive material that connect to the conductive paths to facilitate an electrical connection between the electrical components and module 140 when module 140 is received in dock 120. For example, conductive paths may be connected to conductive terminal pins of dock 120. In one embodiment, dock 120 of knee brace 100 may have a surface that is flush or continuous with the rest of knee brace 100. In one embodiment, dock 120 of knee brace 100 may be a stiffened area of knee brace 100.

As depicted, flexible battery belt 130 may include module 140 for attaching to dock 120, and a belt portion 150 for wrapping around knee brace 100. Belt portion 150 may include a battery source configured to supply power to the electrical components of knee brace 100. In the depicted embodiment, the battery source is not visible since it is covered by an outer fabric layer. In some embodiments, flexible battery belt 130 may wrap around knee brace 100 to provide compression pressure for supporting the muscles of the knee. By providing a power source as flexible battery belt 130, this allows flexible battery belt 130 to have a dual function: powering the electrical components and providing compression pressure.

Module 140 may be received in dock 120 and may be a control module for controlling one or more electrical components of knee brace 100. In some embodiments, module 140 may be attached to dock 120 by magnets. In other embodiments, module 140 may be attached to dock 120 by mechanical latches. In yet other embodiments, module 140 is attached to dock 120 by both magnets and mechanical latches.

The one or more electrical components may not be operational when module 140 is not attached to dock 120. The one or more electrical components connected to dock 120 may become operational when module 140 is attached to dock 120. A battery source may be mechanically and electrically coupled to module 140 and may supply power to the electrical components when module 140 is attached to dock 120. Module 140 may be configured to control the amount of power supplied to the electrical components.

Providing a control module that can be readily removable from dock 120 provides the benefit of controlling when to activate the monitoring or treatment function of knee brace 100, and when to use knee brace 100 without any of its monitoring or treatment functions. Further, providing a battery source separate from knee brace 100 allows for separate handling of knee brace 100 thereby making tasks such as cleaning knee brace 100 easier. Another advantage of providing a battery source separate from knee brace 100 is that it reduces the risk of electric shock to a user. Yet another advantage of providing a battery source separate from knee brace 100 is that it eases the process of charging a battery source and/or replacing a battery source.

In some embodiments, dock 120 and module 140 may include features of the docks and modules disclosed in International Patent Application No. PCT/CA2018/051654, the entire contents of which are herein incorporated by reference.

Flexible battery belt 130 may be attached to elastic strap 160 that wraps around knee brace 100 to provide compression pressure. Flexible battery belt 130 may be attached to elastic strap 160 by buckles, or other fastening means, such as, by being sewn to each other, by hooks, by latches, by snap-fit, by Velcro, by clips, by buttons, or by zippers. In some embodiments, flexible battery belt 130 and elastic strap 160 may be separate and detached from one another. In some embodiments, flexible battery belt 130 may be elastic and/or have an adjustable length.

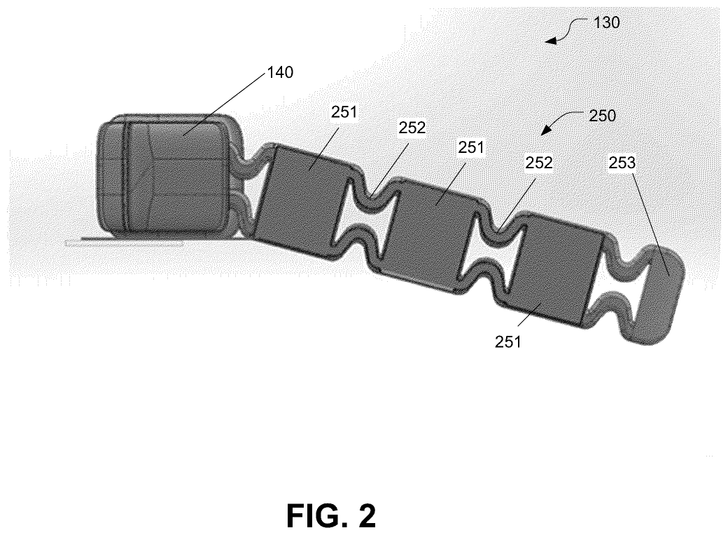

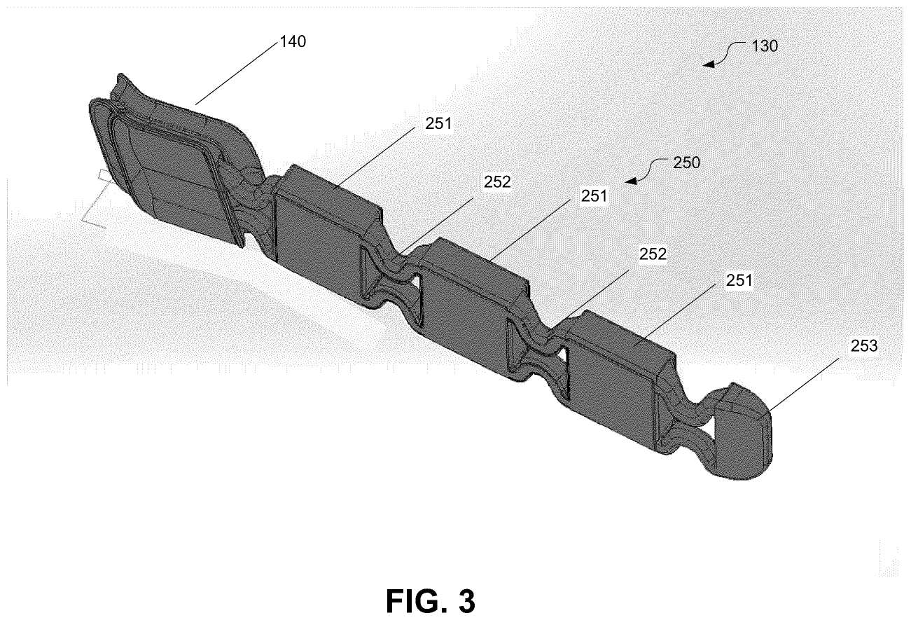

FIGS. 2 and 3 depict a front view and a perspective view, respectively, of flexible battery belt 130 and module 140. As depicted, flexible battery belt 130 may include module 140 electrically coupled to battery assembly 250 made from a plurality of battery units 251 connected by connecting portions 252 to provide flexibility to the belt. End portion 253 of battery assembly 250 may be attached to a fastener coupled to module 140 or may be attached directly to module 140. Battery units 251 may be lithium batteries, alkaline batteries, or biocompatible batteries for use in proximity to a human body. Although it is not depicted, battery assembly 250 may be included in belt portion 150 in FIG. 1. Battery assembly 250 may be covered with an outer tubing, such as a fabric outer tubing to be belt portion 150 as illustrated in FIG. 1.

Although FIGS. 2 and 3 depict flexible battery belt 130 including 3 battery units, it should be appreciated that battery belt 130 may include a different number of battery units. For example, flexible battery belt 130 may include one battery unit, two battery units, four battery units, or five or more battery units. Although FIGS. 2 and 3 depict flexible battery belt 130 including a battery units 251 in series, it should be appreciated that battery units 251 may be arranged in parallel.

Battery units 251 may be connected by wires and housed in an elastomeric material, such as plastic, to form elongated battery assembly 250 that is flexible. Battery assembly 250 may be axially and/or longitudinally bendable. In some embodiments, battery assembly 250 may be extendable.

As depicted in FIGS. 2-3, connecting portions 252 between adjacent battery units may be curved or bent to allow for flexibility and movement between battery units 251. In some embodiments, connecting portion 252 between each battery unit may be foldable to allow for flexibility and movement between battery units.

In some embodiments, each battery unit of battery units 251 may be attached to an adjacent battery unit by one connecting portion. In other embodiments, each battery unit of battery units 251 may be attached to an adjacent battery unit by two or more connecting portions. In an alternative embodiments, battery units 251 may be soldered to a flexible printed circuit board (PCB).

Battery assembly 250 may be electrically coupled to module 140 at a first end of battery assembly 250. Elongated battery assembly may also include second end 253 that is electrically coupled to module 140. Second end 253 may also be mechanically coupled to module 140 to anchor second end 253. In other embodiments, second end 253 of battery assembly 250 may only be mechanically coupled to module 140 and not electrically coupled to module 140. In another embodiment, second end 253 of battery assembly 250 may be attached to a fastener that attaches to elastic strap 160 for wrapping around knee brace 100.

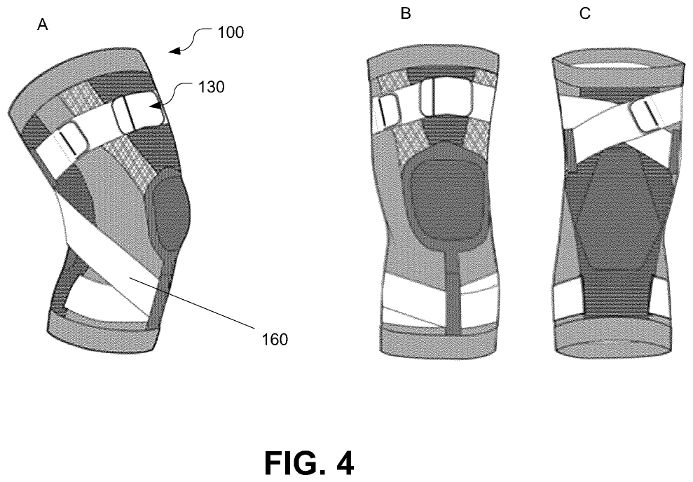

FIG. 4 depicts a perspective view A, front view B and rear view knee C of knee brace 100 including flexible battery belt 130 and elastic strap 160.

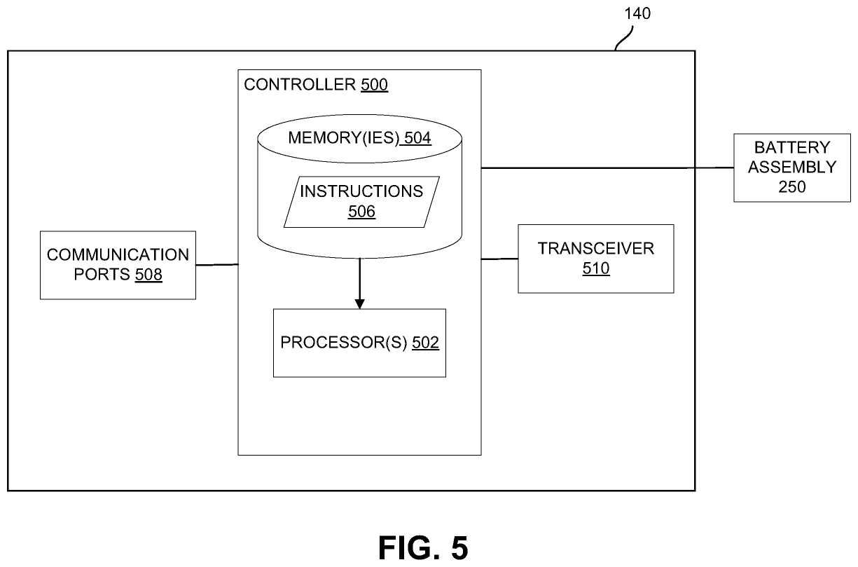

FIG. 5 depicts a schematic depiction of module 140 including computing device 500, one or more communication ports 506 for receiving and transmitting electrical signals, and transceiver 510 for wirelessly transmitting data. In alternative embodiments, computing device 500 may be coupled to module 140. Computing device 500 may include one or more data processors 502 (referred hereinafter in the singular) and one or more computer-readable memories 504 (referred hereinafter in the singular) storing machine-readable instructions 506 executable by data processor 502 and configured to cause data processor 502 to generate one or more outputs (e.g., signals) for causing the execution of one or more steps of the methods described herein.

Data processor 502 may include any suitable device(s) configured to cause a series of steps to be performed by computing device 500 so as to implement a computer-implemented process such that instructions, when executed by computing device 500 or other programmable apparatus, may cause the functions/acts specified in the methods described herein to be executed. Data processor 502 may include, for example, any type of general-purpose microprocessor or microcontroller, a digital signal processing (DSP) processor, an integrated circuit, a field programmable gate array (FPGA), a reconfigurable processor, other suitably programmed or programmable logic circuits, or any combination thereof.

Memory 504 may include any suitable machine-readable storage medium. Memory 504 may include non-transitory computer readable storage medium such as, for example, but not limited to, an electronic, magnetic, optical, electromagnetic, infrared, or semiconductor system, apparatus, or device, or any suitable combination of the foregoing. Memory 504 may include a suitable combination of any type of computer memory that is located either internally or externally to computing device 500. Memory 504 may include any storage means (e.g. devices) suitable for retrievably storing machine-readable instructions 506 executable by data processor 502.

Various aspects of the present disclosure may be embodied as systems, devices, methods and/or computer program products. Accordingly, aspects of the present disclosure may take the form of an entirely hardware embodiment, an entirely software embodiment or an embodiment combining software and hardware aspects. Furthermore, aspects of the present disclosure may take the form of a computer program product embodied in one or more non-transitory computer readable medium(ia) (e.g., memory 504) having computer readable program code embodied thereon. Computer program code for carrying out operations for aspects of the present disclosure in accordance with instructions may be written in any combination of one or more programming languages. Such program code may be executed entirely or in part by computing device 500 or other data processing device(s). Based on the present disclosure, one skilled in the relevant arts could readily write computer program code for implementing the methods described herein.

In some embodiments, module 140 may receive signals from a sensor of knee brace 100 via communication ports 506. In response to receiving the signal, computing device 500 may be configured to transmit data indicative of the sensor reading to a remote server via transceiver 508. The data may be stored and/or analysed. In another example, computing device 500 may be configured to wirelessly communicate with other modules or devices, such as a mobile device, to allow a user to continuously monitor physiological data pertaining to the user's knee.

Computing device 500 may include interfaces, including application programming interfaces, and network communications interfaces. For example, computing device 500 may interconnect with a message bus or other type of data bus for shared communications and data messaging, which may be synchronized to a common clock element. Computing device 500 may include a wireless communication interfaces such as Bluetooth® (including Bluetooth® Low Energy) or the like, for sending and receiving data.

Although it is not depicted in FIG. 4, module 140 may include control buttons and/or indicators. In some embodiments, module 140 has LED indications for the status of the one or more electrical components. For instance, module 140 may include one or more LEDs for indicating the status of a stimulator that provides electrical stimulation to at least a part of the knee. In some embodiments, module 140 may include control buttons for activating one or more electrical components. For example, a control button may be disposed on module 140 and when depressed by a user may facilitate electrical power to be supplied from a power source, such as battery assembly 250, to an electrical component of knee brace 100.

In some embodiments, transceiver 508 may be a Bluetooth® transceiver. The Bluetooth® transceiver may be used for sending data to a mobile phone for example. In some embodiments, module 140 may include a micro usb port for charging or data transfer, a current sensor, an IMU (inertial measurement unit), a memory, a temperature sensor, a haptic motor, a heat boost converter, an electrical stimulation boost converter, a heat driver, an electrical stimulation driver, or combinations thereof.

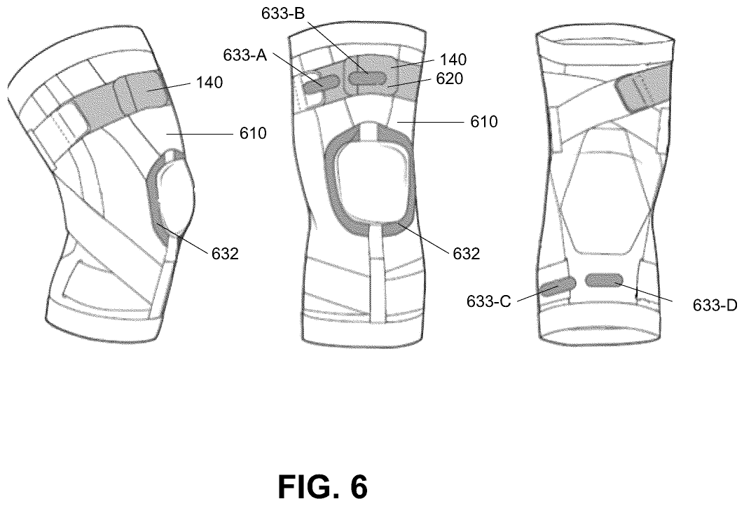

FIG. 6 depicts an example embodiment of knee brace 100 including heating element 632 and stimulators 633-A-D (hereinafter referred to as stimulators 633) connected to dock 620. Module 140 may be received in dock 620 to activate the electrical components. In reference to FIG. 1, some elements included in knee brace 100 depicted in FIG. 1 are common to knee brace 100 depicted in FIG. 6. Reference character of like elements have been incremented by 500 and their description is not repeated.

Heating element 632 may provide heat to at least a part of the knee of the user. Heating element 632 may be inlaid in textile article 610. In some embodiments, heating element 632 is positioned to at least partially surround or cover the knee cap when knee brace 100 is worn by the user. In some embodiments, heating element 632 is positioned to encircle the anterior of the kneecap.

Heating element 632 may be formed of conductive yarn inlaid within textile article 610 that is arranged to provide resistive heating. Heating element 632 may form part of textile article 610. Heating element 632 may be electrically coupled to module 140 by way of one or more conductive paths defined by conductive yarn of textile article 610 when module 140 is received in dock 620. The one or more conductive paths may lead from power terminal(s) of heating element 632 to power terminal(s) of dock 620. Heating element 632 may receive power from a battery assembly electrically coupled to module 140, such as battery assembly 250 for example, to heat heating element 632. It is understood that other materials or devices may be used for providing heat to the knee.

Stimulators 633 may be formed of an electrode acting as a transducer in converting the ionic current in/on the body into electron currents in conductive wires and electronic circuits, and vice versa.

An electrode may generally be defined as conductive material through which an electrical current passes to a body of a user and/or a voltage is received from the body of a user. An electrode can function as a sensor when receiving electrical energy for measurement/recordation. An actuator can function as an actuator when injecting electrical current/voltage to the body, e.g. for FES to inject electrical pulses to activate muscles.

Stimulators 633 may be formed of a dry contact electrode. Dry contact electrodes can be categorized according to form factor into textile electrodes, flexible film electrodes, bulk electrodes, pin-shaped electrodes, and microneedles. Dry electrodes may be biocompatible, easy to use, comfortable, breathable, lightweight, flexible, washable, durable, and able to maintain good signal quality during electrophysiology testing while at rest and moving. Additionally, textile-based electrodes may be worn on various body parts by attaching them to different articles of clothing such as waistbands, sleeves, pants, headbands, etc.

Dry contact electrodes may be more convenient than standard wet gel electrodes in some respects. For example, standard electrodes may use an electrolytic gel to maintain good electrical contact with the Stratum Corneum, creating an ionic path between the electrode and the skin below the Stratum Corneum via conductive ions in the gel. This reduces the skin impedance and allows for improved signal acquisition. However, the standard wet gel electrode used currently, e.g. in healthcare, may have limitations. The adhesive can cause skin irritation and becomes uncomfortable over time, the gel dehydrates with time thus degrading signal quality, and the electrode can be uncomfortable to the user, due to its metallic piece, therefore a soft, textile form is an inconspicuous alternative for continuous health monitoring.

In some embodiments, simulators 633 may be dry contact, textile-based electrodes, as disclosed for example in U.S. patent application Ser. No. 62/955,546, entitled “CONDUCTIVE THERMOPLASTIC ELASTOMER ELECTRODES, AND METHOD OF MANUFACTURING SUCH ELECTRODES”, the entire contents of which are herein incorporated by reference.

Stimulators 633 may provide stimulation to a muscle or a nerve of the knee of a user. In some embodiments, stimulators 633 may be transcutaneous electrical nerve stimulators configured to transmit low-level electric current to a muscle or nerve of the knee. The stimulators may be located at various positions in knee brace 100, preferably in close proximity to a target area of the knee. A target area of the knee may be, but is not limited to, a knee joint, a knee cap, a knee or leg muscle, a blood vessel, a nerve or a nerve ending, or a bone.

Each of stimulators 633 may be electrically coupled to module 140 by way of one or more conductive paths defined by textile article 610 when module 140 is received in dock 620. The one or more conductive paths may lead from power terminal(s) of stimulator 633 to power terminal(s) of dock 620. Stimulators 633 may receive power from a battery assembly electrically coupled to module 140, such as battery assembly 250 for example, to allow stimulator 633 to provide electrical stimulation to the knee.

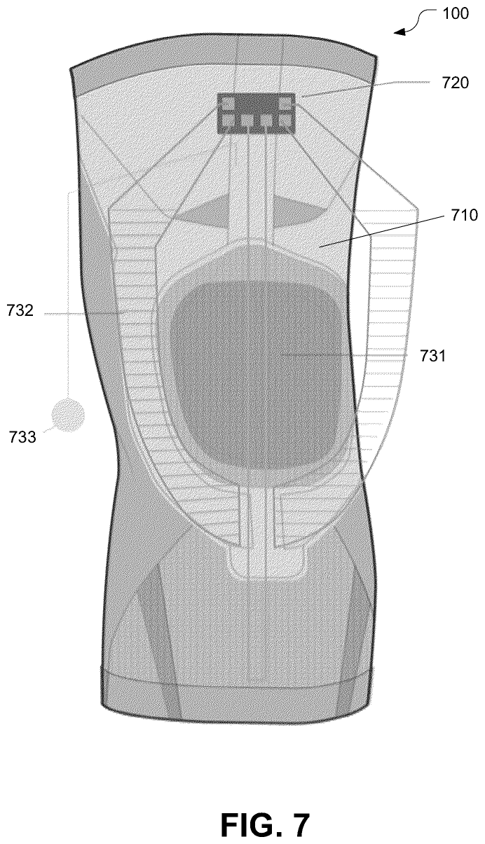

As depicted in FIG. 7, in addition to the electrical components included in the embodiment of FIG. 6, knee brace 100 may also include stretch sensor 731. Although module 140 is omitted in FIG. 7, module 140 may be received in dock 720 to activate the electrical components. In reference to FIG. 6, some elements included in knee brace 100 depicted in FIG. 6 are common to knee brace 100 depicted in FIG. 7. Reference character of like elements have been incremented by 100 and their description is not repeated.

Stretch sensor 731 may be used for detecting orientation and changes in orientation, thereby permitting the monitoring of movement, such as knee bends, muscle contraction, or other movements involving the knee . Stretch sensor 731 may be used for detecting and monitoring knee flexion and/or extension and/or knee varus-valgus motion and/or gait. Stretch sensor 731 may be used for detecting and/or monitoring steps, travel distance, falls, knee load, or muscle strength. It should be understood that one or more different types of sensors, such as a pair of inertial measurement units (IMU) (As shown in FIG. 4), may be used in conjunction with stretch sensor 731 or may be used on their own to detect and monitor these parameters.

Stretch sensor 731 may be inlaid in textile article 710 of knee brace 100. Stretch sensor 731 may be electrically coupled to module 140 by way of one or more conductive paths defined by textile article 710 when module 140 is received in dock 720. The one or more conductive paths may lead from power terminal(s) of stretch sensor 731 to power terminal(s) of dock 720. Stretch sensor 731 may receive power from a battery source, such as battery assembly 250, electrically coupled to module 140. Stretch sensor 731 may transmit signals indicative of a measurement recorded by the stretch sensor 731 to module 140 via the one or more conductive paths.

Stretch sensor 731 may be oriented vertically or horizontally in knee brace 100. Different orientations of sensor 731 in knee brace 100 may result in different measurement readings. In some embodiments, stretch sensor 731 of knee brace 100 may be positioned to vertically span over the anterior of the knee. In some embodiments, stretch sensor 731 spans 2 inches above and 2 inches below the kneecap. In alternate embodiments, stretch sensor 731 spans about 1 inches above and below the kneecap, about 1.5 inches above and below the kneecap, about 2 inches above and below the kneecap, about 2.5 inches above and below the kneecap, about 3 inches above and below the kneecap, or more.

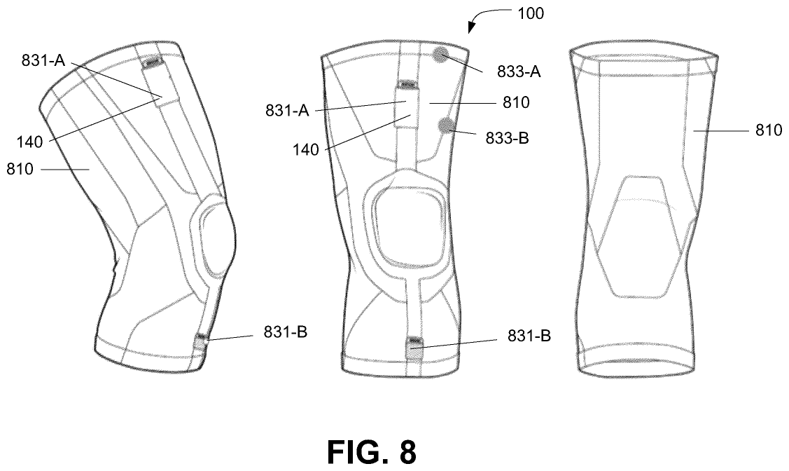

FIG. 8 depicts a perspective, front view and rear view of another example embodiment of knee brace 100 including electrical components (a pair IMUs 831-A and 831-B and sensors 832-A and 832-B). Although the electrical components are visible in the illustration in FIG. 8, it should be understood that the electrical components may be inlaid within textile article 810 and therefore not visible once knee brace 100 is manufactured. Module 140 and battery source may be integrated with IMU 831-A to control and provide power to the electrical components. In reference to FIG. 1, some elements included in knee brace 100 depicted in FIG. 1 are common to knee brace 100 depicted in FIG. 8. Reference character of like elements have been incremented by 700 and their description is not repeated. In this embodiment, knee brace 100 may not include a flexible battery belt or dock as described in FIG. 1. This may be due to lower power requirements of the electrical components.

As depicted in FIG. 8, IMU 831-A may be positioned at a first location in knee brace 100. IMU 831-B may be positioned at a second location in knee brace 100. The first position may be different than the second position. In some embodiments, the first position may be above a kneecap of the knee and the second position may be below the kneecap of the knee. Each IMU includes a three-axis accelerometer, a gyroscope and a magnetometer.

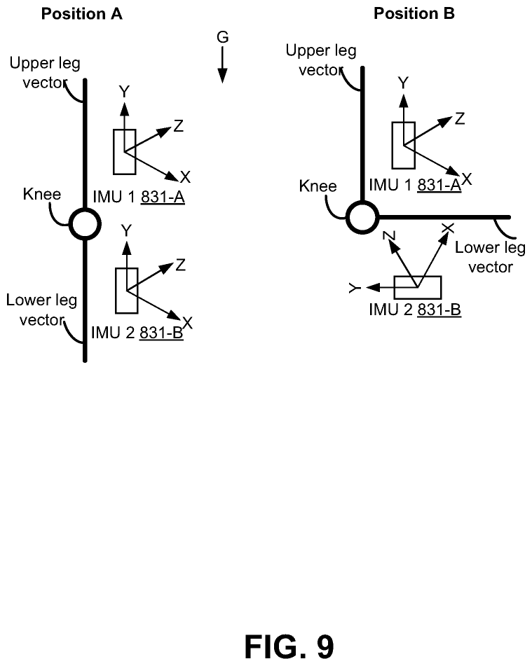

When knee brace 100 is in use by a wearer, having at least one IMU above the kneecap of the knee and at least one IMU below the kneecap of the knee, may enable module 140 or a remote computer in communication with module 140 to determine an orientation of the knee. A difference between an acceleration measurement reading of IMU 831-A located above the kneecap and an acceleration measurement reading of IMU 831-A located below the kneecap may be used to determine the physiological state of the knee. For example, FIG. 9 shows the orientation and axis of IMUs 831-A and 831-B of knee brace 100 worn by a wearer when the wearer's knee is at two different positions, position A and position B. Position A illustrates a case when the wearer's knee is completely straight. Position B illustrates a case when a lower leg of the wearer is bent at a 90 degree angle relative to the upper leg of the wearer. At position A, the Y-axis acceleration reading of both IMU 831-A and IMU 831-B may be the same (as a consequence of gravity G). However, at position B, the Y-axis acceleration reading for IMU 831-A may be different than the Y-axis acceleration reading for IMU 831-B. The Y-axis acceleration reading for IMU 831-B may be zero, while the Y-axis acceleration reading for IMU 831-A may be the same as the reading of IMU 831-A in position A. The difference between the readings of IMU 831-A and IMU 831-B may be used to compute the physiological state of the knee of the wearer.

The pair of IMUs 831-A and 831-B may be used for detecting orientation and changes in orientation, thereby permitting the monitoring of movement, such as knee bends, muscle contraction, or other movements involving the knee. The pair of IMUs 831-A and 831-B may be used for detecting and monitoring knee flexion and/or extension and/or knee varus-valgus motion and/or gait. The pair of IMUs 831-A and 831-B may be used for detecting and/or monitoring steps, travel distance, falls, knee load, or muscle strength.

The pair of IMUs 831-A and 831-B may be inlaid in textile article 810 of knee brace 100. Each IMU may be electrically coupled to module 140 by respective conductive paths defined by textile article 810. One or more conductive path may lead from power terminal(s) of a given IMU to power terminal(s) of module 140. The pair of IMUs 831-A and 831-B may receive power from a battery source electrically coupled to module 140. The pair of IMUs 831-A and 831-B may each transmit signals indicative of inertial measurements to module 140.

Sensors 832-A and 832-B may be used for measuring the electrical activity of muscle of the knee at rest and during contraction. In some embodiments, sensors 832-A and 832-B may be dry electrodes as previously described. In some embodiments, sensors 832-A and 832-B may be EMG sensors that are inlaid in textile article 810 of knee brace 100. Sensors 832-A and 832-B may be electrically coupled to module 140 by respective conductive paths defined by textile article 810. One or more conductive paths may lead from power terminal(s) of a given sensor to power terminal(s) of module 140 (not depicted). Sensors 832-A and 832-B may receive power from a battery source electrically coupled to module 140. Sensors 832-A and 832-B may transmit signals indicative of the measured electrical activity to module 140.

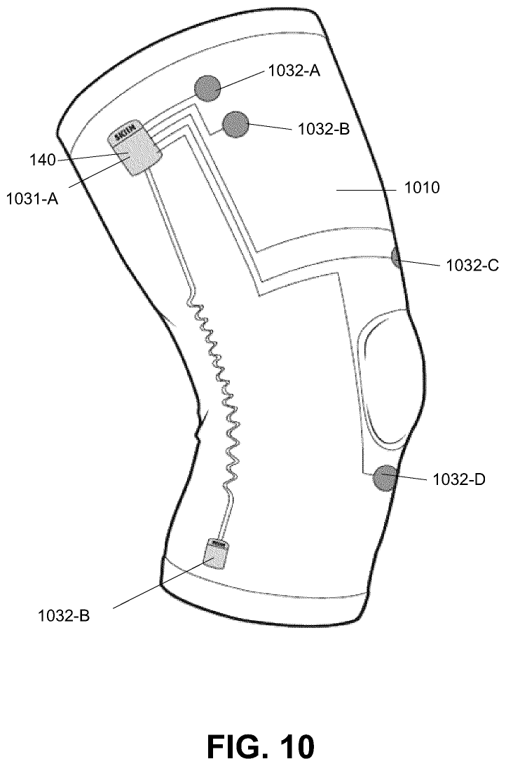

FIG. 10 depicts a perspective of another example embodiment of knee brace 100, similar to the embodiment depicted in FIG. 8, including electrical components (a pair IMUs 1031-A and 1031-B and EMG sensors 1032-A-D). Module 140 and battery source may be integrated with IMU 1031-A to control and provide power to the electrical components. In this embodiment, conductive paths between the electrical components and module 140 are clearly depicted. Although the electrical components and conductive paths are visible in the illustration in FIG. 10, it should be understood that the electrical components and conductive paths may be inlaid within textile article 1010 and therefore not visible once knee brace 100 is manufactured. In reference to FIG. 8, some elements included in knee brace 100 depicted in FIG. 8 are common to knee brace 100 depicted in FIG. 10. Reference character of like elements have been incremented by 200 and their description is not repeated.

As depicted, IMU 1031-B may be electrically coupled to module 140 by respective conductive paths defined by textile article 1010. One or more conductive path may lead from power terminal(s) of IMU 1031-B to power terminal(s) of module 140. The pair of IMUs 1031-A and 1031-B may receive power from a battery source electrically coupled to module 140. The pair of IMUs 1031-A and 1031-B may each transmit signals indicative of inertial measurements to module 140.

As depicted, sensors 1032-A-D may be electrically coupled to module 140 by respective conductive paths defined by textile article 1010. One or more conductive paths may lead from power terminal(s) of a given sensor to power terminal(s) of module 140. Sensors 1032-A-D may receive power from a battery source electrically coupled to module 140. Sensors 1032-A-D may transmit signals indicative of the measured electrical activity of a muscle of the knee to module 140.

In embodiments where a flexible battery belt having a module is attached to the knee brace, attaching the module to the dock also provides electrical power to the one or more electrical components. In some embodiments, a user then wraps the battery belt around the knee brace and adjusts the lengths as needed to create desired compression pressure. In other embodiments, a user attaches the battery belt to an elastic strap for wrapping around the knee brace to achieve desired compression pressure. Alternatively, the battery belt is attached to the elastic strap first prior to attaching the module to the dock.

In embodiments where the knee brace has a heating element, the heating element is selectively activated at desired times to provide heat to the knee. For example, a user can selectively activate the heating element prior to exercise to warm up the knee joint and/or knee muscles. For example, a user can activate the heating element to improve circulation or to reduce stiffness of a target area of the knee. In some embodiments, activating the heating element comprising providing heat therapy to a target area of the knee.

In embodiments where the knee brace has a stimulator, such as an electrode, the stimulator is selectively activated at desired times to provide stimulation to a nerve or muscle. For example, a user can activate the stimulator to relieve muscle soreness or knee pain. In some embodiments, activating the stimulator comprising providing transcutaneous electrical nerve stimulation (TENS) therapy to a target area of the knee.

Numerous details are set forth to provide an understanding of the examples described herein. The examples may be practiced without these details. The description is not to be considered as limited to the scope of the examples described herein.

Although the embodiments have been described in detail, it should be understood that various changes, substitutions and alterations can be made herein. Moreover, the scope of the present application is not intended to be limited to the particular embodiments or examples described in the specification. As can be understood, the examples described above and illustrated are intended to be exemplary only.

For example, the present invention contemplates that any of the features shown in any of the embodiments described herein, may be incorporated with any of the features shown in any of the other embodiments described herein, and still fall within the scope of the present invention.

What is claimed is:

1. A knee brace for monitoring a knee of a wearer, the knee brace comprising:
a textile article shaped to cover at least part of a knee of the wearer, the textile article including conductive yarn arranged to define a first conductive path;
a dock for removably receiving a control module, the control module electrically coupled with the dock when received therein;
a sensor for measuring a physiological state of the wearer, the sensor being electrically coupled to the control module by way of the first conductive path when the control module is received in the dock.

2. The knee brace of claim 1, wherein the sensor is inlaid within the textile article.

3. The knee brace of claim 1, wherein:
the sensor is a stretch sensor configured to detect and monitor movement of the knee; and
the stretch sensor extends from above a kneecap of the knee to below the kneecap of the knee.

4. The knee brace of claim 1, wherein:
the sensor is a first sensor and the knee brace includes a second sensor;
the first sensor and the second sensor are accelerometers configured to detect an orientation of the knee;
the first sensor is disposed at a first position and the second sensor is at a second position away from the first position.

5. The knee brace of claim 4, wherein the first location is above a kneecap of the knee and the second location is below the kneecap of the knee.

6. The knee brace of claim 1, wherein:
the control module is electrically coupled to a battery assembly that powers the sensor; and
the control module and the battery assembly form a flexible belt configured to wrap around the textile article when the textile article is worn by the wearer.

7. The knee brace of claim 6, wherein the battery assembly includes a plurality of battery units housed in an elastomeric material, each battery unit of the plurality of battery units being connected to an adjacent battery unit of the plurality of battery units by a flexible connecting member.

8. The knee brace of claim 6, wherein the flexible belt is attached to an elastic strap that wraps around the textile article and provides compression pressure when the textile article is worn by the wearer.

9. The knee brace of claim 1 further comprising:
a heating element inlaid within the textile article for providing heat to at least a part of the knee;
wherein, when the control module is received in the dock, the heating element is electrically coupled to the control module by a second conductive path defined by the conductive yarn that extends between the heating element and the dock.

10. The knee brace of claim 9, wherein the heating element is a third conductive path arranged to provide resistive heating.

11. The knee brace of claim 1 further comprising:
a stimulator integrated within the textile article for providing stimulation to a nerve or muscle of the knee;
wherein, when the control module is received in the dock, the stimulator is electrically coupled to the control module by a third conductive path defined by the conductive yarn that extends between the stimulator and the dock.

12. The knee brace of claim 11, wherein:
the stimulator includes an electrode configured to provide electrical stimulation to a target area of the knee; and
when the knee brace is worn by the wearer, the stimulator is positioned proximate the target area of the knee.

13. The knee brace of claim 11, wherein the stimulator is a transcutaneous electrical nerve stimulator.

14. The knee brace of claim 1 further comprising:
at least one electromyography (EMG) sensor for measuring electric potential generated by a muscle of the knee;
wherein, when the control module is received in the dock, the EMG sensor is electrically coupled to the control module by a second conductive path defined by the conductive yarn that extends between the EMG sensor and the dock.

15. The knee brace of claim 1, wherein the control module includes:
a communication port;
a transceiver;
one or more processors; and
non-transitory machine-readable memory storing instructions executable by the one or more computer processors and configured to cause the one or more computer processors to:
receive via the communication port from the sensor data indicative of the measured physiological state of the knee of the wearer; and
transmit the data via the transceiver to a remote server which stores the data.

16. The knee brace of claim 15, wherein the transceiver is a Bluetooth® transceiver.

17. The knee brace of claim 1, wherein the control module is secured to the dock using magnets.

18. The knee brace of claim 1, wherein the control module is secured to the dock using a mechanical latch.
